(19) United States
(12) Patent Application Publication
(10) Pub. No.: US 2026/0053503 A1
(43) Pub. Date: Feb. 26, 2026

(54) CONNECTION CABLE AND DETACHMENT DEVICE

(71) Applicant: STANDARD INSTRUMENTS GMBH GESELLSCHAFT FÜR MEDIZINTECHNIK, MESS- UND REGELTECHNIK, STEUERUNGSTECHNIK, Karlsruhe (DE)

(72) Inventors: Andreas SCHÖNFELD, Karlsruhe (DE); Patrik FÖLLMER, Karlsruhe (DE)

(73) Assignee: STANDARD INSTRUMENTS GMBH GESELLSCHAFT FÜR MEDIZINTECHNIK, MESS- UND REGELTECHNIK, STEUERUNGSTECHNIK, Karlsruhe (DE)

(21) Appl. No.: 18/996,320

(22) PCT Filed: Jul. 18, 2023
(86) PCT No.: PCT/EP2023/069949

§ 371 Date: Jan. 17, 2025

(30) Foreign Application Priority Data
Jul. 18, 2022 (DE) 10 2022 117 911.7

(51) Int. Cl. A61B 17/12; H01R 13/631

(52) U.S. Cl. CPC A61B 17/12113; A61B 17/12145; H01R 13/631; A61B 2017/12063; A61B 2017/12068; H01R 2201/12

(57) ABSTRACT

The invention relates to a cable for the connection of an electrical detachment device to the end of a catheter-guided implant, with the cable comprising at least two current conducting wires, a connecting plug for joining the cable to the detachment device, and a plug connector for connection to current conducting elements of the implant, and wherein the current conducting wires being connected on the device side to con-tacts of the connecting plug and on the implant side to contacts of the plug connector, characterized in that the cable on the implant side being provided with an insertion funnel which is suitable for conductively joining the current carrying elements of the implant to the contacts of the plug connector.

The invention relates to a cable for connecting an electrical detachment device to the end of an implant which is guided by catheter, a device for detaching a catheter-guided implant from a guidewire, as well as a combination comprising detachment device and connection cable.

Catheter-guided implants are widely used for the treatment of vascular defects. Aneurysms are, for example, accessed endovascularly via a catheter and occluded by means of a catheter-guided implant, such as a spiral consisting of a noble metal alloy. The implant is arranged at the tip of a guidewire to which it is connected by a detachment element. Such a detachment element is, for example, a metal element that can be dissolved by electrolysis or a plastic element that can be thermally melted off. In the event of electrolysis, the implant is detached from the guidewire by applying a weak electric current, usually in the range of between 1 mA and 2.5 mA. The detachment time ranges between 0.5 and 10 sec. When thermal detachment processes are used, a higher current is applied, typically in the range of between 100 and 300 mA, with the detachment times as a rule being less than 1 second.

In case the detachment is brought about electrolytically, the electrical current is conducted from the detachment device via the guidewire to the implant. The electrical circuit can be established either via a wire arranged within the catheter or via a back electrode situated on the patient's body. In the former case, a conductive core is provided within the guidewire and insulated against it, said core carrying the back current; in the latter case, the back current is conducted via a needle that is placed in the patient's shoulder area and has a cable connection to the detachment device. On the one hand, the back current flow that takes place via the needle is associated with an injury, albeit a minor one, but on the other hand, it also possibly results in the connection characteristics for the back current to deteriorate because varying body resistances will be involved causing the overall resistance in the system to increase.

Due to the fact that the current involved in thermal detachment is usually much higher, current return via the body is not advisable here. Rather, the thermal current in this case is conducted directly in the guide system via lines. Systems are known that are provided with one or two insulated wires arranged within the guidewire.

Two basic principles are known for the electrical connection of the placement systems with or without a needle. On the one hand, there are the systems in which the guidewire constitutes one pole and the needle the other, so-called monopolar systems, while, on the other hand, there are the so-called bipolar systems in which both poles are concentrically arranged on the guidewire.

The implants and the technique for their detachment and deposition in a patient's body are known. For example, implants are commercialized under the brand names Matrix, GDC and Target. The respective implant designs each have special detachment requirements that must be met by the detachment devices used. In particular, such detachment requirements notably include current intensity and detachment time. The detachment devices are appropriately adapted to the individual implant designs. The systems also make appropriate allowances for the different contactability of the device-side ends of the implant systems.

Contact-making at the respective connection points is achieved using terminals or spring contacts, which usually have a gold layer and are subjected to mechanical stress. This means that they can be reused to a limited extent only. Although these contact arrangements do not come into direct contact with the patient when used as intended, they may still be contaminated with blood, for example. In any case, their application is limited to using them on a single patient only, since also a resterilization is associated with risks, high costs and an uncertainty regarding the safety of the contacts.

Since the patient's treatment area must be kept sterile, the same requirement for sterility applies to devices used in this area. This is a problematic issue due the fact that the process of resterilizing the devices is very complex and involves a great deal of residual risk, so the common practice is to dispose of the devices after each patient treatment. Apart from the high costs involved, this is also to be considered a waste of valuable resources.

This applies in particular to devices such as those that are described in US 2009/018 653 A1 and are presently also put to use. These are hand-held devices that can be used for both monopolar and bipolar systems. The devices are provided with a connection shaft. Since these devices are monitored and operated by the attending physician, they are necessarily implemented in the sterile area, so that, for second use, a comprehensive sterilization is required, since contamination by the previously treated patient cannot be ruled out. Such sterilization procedure, if at all possible in the area of the connection shaft, is extremely elaborate and costly which therefore results in the devices being disposed of after single use.

For electrolytically based detaching systems, it has become standard practice to operate the electronic detachment device outside the sterile area and to use only a connecting line leading from the device to the implant's guidewire and to the needle. This connecting line is used in sterile condition and applied for each new patient. However, the expensive electronic unit is reused because it is located outside the sterile area.

Such a solution does not exist for bipolar systems; thermal detachment systems as well are equipped with bipolar contacts and require a device for each patient.

Therefore, objective of the invention is to provide a system that allows detachment devices to be located and operated external to the sterile treatment area, so that the system can be reused without sterilization being required. It would also be desirable to make use of a universal detachment device for the variety of implants currently available on the market.

This objective is achieved by means of a cable for connecting an electrical detachment device to the end of a catheter-guided implant, with the cable comprising at least two current conducting wires, a connecting plug for joining the cable to the detachment device, and a plug connector for connection to current conducting elements of the implant, and with the current conducting wires being connected on the device side to contacts of the connecting plug and on the implant side to contacts of the plug connector, and wherein on the implant side the cable is provided with an insertion funnel which is suitable for conductively joining the current carrying elements of the implant to the contacts of the plug connector. If appropriately dimensioned, a cable of this type is suitable for all bipolar connection systems.

In the context of the present invention, the term “device side” refers to the end of the cable intended for connection to the detachment device and the term “implant side” refers to the end of the cable intended for connection to the implant. In the broader sense, the term “implant” shall be understood to denote the combination of the actual implant at the end of the guidewire and the guidewire proper, with guidewire and implant being connected to each other via the detachment element. In the narrower sense, the implant is to be seen as the final element of the combination consisting of implant and guidewire that is intended to remain in the patient's body. The cable proposed by the invention is thus intended for establishing a connection between the detachment device and the device-side end of the guidewire of the combination comprising implant and guidewire.

The inventive cable is provided with at least two current conducting wires that can be connected to the detachment device via the connecting plug. On the implant side, these wires terminate in a plug connector that serves to establish a connection to the device-side end of the implant—i.e. the guidewire. Expediently, the contacts required for this purpose are contact springs which are suitably arranged on a printed circuit board and are accessible through an insertion funnel at the implant end of the cable. Such a printed-circuit board, for example, is integrally connected to the insertion funnel and is equipped with all the necessary contacts, circuit traces, etc.

It is advisable for such cables to comprise a remote trigger that allows the treating physician to activate the detachment of the implant. This is advisable, since the detachment device is located outside the sterile treatment area. As a rule, a remote trigger of this kind requires at least a third wire inside the cable as well as the relevant connection elements on the circuit board.

To facilitate the insertion of the device-side end of the implant, it is expedient to arrange for the insertion funnel to have a diameter that is conducive to the dimensions of the implant's current carrying elements. It is also recommendable to tension the contact springs such that they make reliable contact with the conductive elements of the implant system. If considered necessary, a stop can be provided to limit the insertion depth of the current-conducting elements.

Expediently, the connecting plug of the cable according to the invention is provided with guide elements to ensure correct connection to the detachment device as well as latching elements to establish a firm attachment to the device.

Moreover, the invention also relates to a detachment device for the release of a catheter-based implant from a guidewire, wherein the detachment device is provided with a power source, an on/off switch, a release button, signal lights to indicate the operating state and malfunctions, an electronic control unit for the detachment function as well as a connector socket for connection to a cable that establishes a connection to the guidewire of the catheter-guided implant, wherein the connector socket has at least two contacts, latching elements ensuring a firm connection with complementary latching elements of the cable, and insertion elements to make sure the cable has been correctly fitted.

The detachment device proposed by the present invention is conveniently operated by batteries, but it can also be connected to the mains.

The release button serves to separate the implant from the guidewire. Independently of the release button arranged on the detachment device, a release can also be initiated by a remote triggering device in the cable that establishes connection to the implant.

Such detachment devices with their individual functions are basically known. This applies in particular to the individual functions and the electronic control system. The special feature here concerns the connector socket design and configuration, which permits use with a plurality of detachment systems. In particular, the signal lights indicate the operating status, that the operating voltage has been reached, the detachment process has been triggered, the successful detachment of the implant, when detachment has failed, the battery is too weak, the connection cable has been plugged in incorrectly, etc.

The connector socket is preferably a round socket comprising at least three contacts and, in particular, five contacts.7 Contacts in the form of contact pins arranged on the detachment device are preferred, however the contact pins may also be located on the connecting plug of the connection cable.

The latching elements provided on the connector socket serve to fix the cable connecting plug in place on the detachment device. Expediently, the connector socket is provided with two latching elements located opposite each other. It shall be understood that the latched components of connecting plug and connector socket can be disengaged again, for example by pulling gently or using another disconnecting mechanism.

Moreover, the detachment device has guide elements that ensure the connecting plug is inserted correctly. Especially if multipolar plug connectors are employed, it is possible to provide appropriate electrical coding via two or more contacts to communicate to the device which implant is to be used. In this way, the appropriate settings can be made for the desired detachment parameters. The plug of the implant-specific connection cable thus determines the functionality of the device. Also feasible is a purely mechanical coding mechanism. Two guide elements, for example in the form of guiding lugs, that are spaced apart and arranged between the latching elements, allow the connection of at least three different connecting plugs for three different detachment systems. Inserting the plug in the correct position will then simultaneously activate the correct detachment current as well as the correct time period for safe detachment.

Finally, the invention also relates to the combination of the detachment device described hereinbefore with the respective cables of a variety of detachment systems, in particular, however, with the detachment system for bipolar implants.

The invention is explained in more detail by way of the following figures showing preferred embodiments. Clarification of the invention is provided by the figures below, where

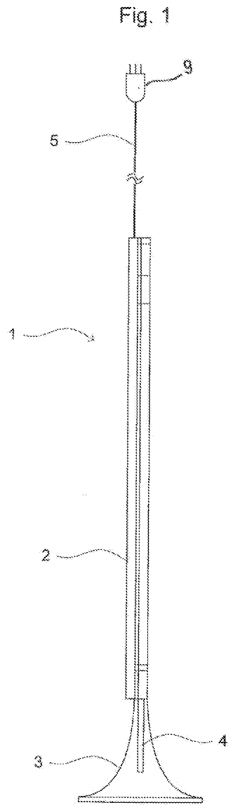

FIG. 1 shows a lateral view of the implant-side end of a cable according to the invention;

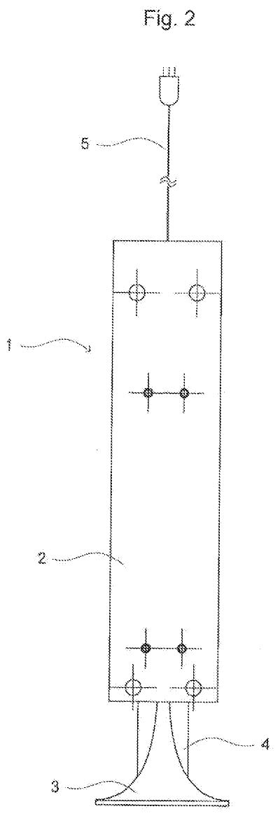

FIG. 2 shows a top view of the implant-side end of a cable according to the invention;

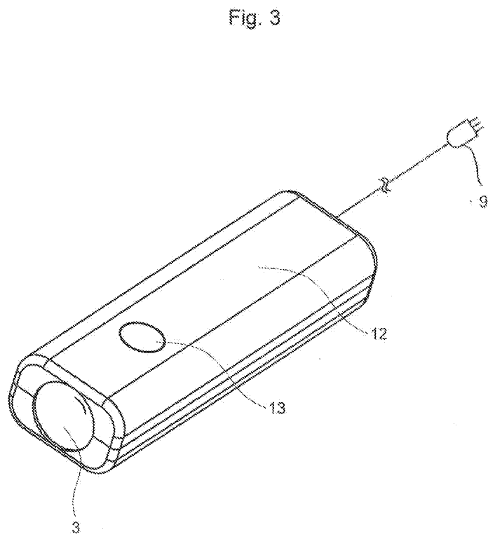

FIG. 3 illustrates the implant-side end of the cable with enclosure according to the invention;

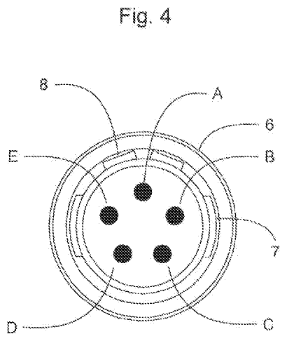

FIG. 4 shows a connector socket of a detachment device proposed by the invention;

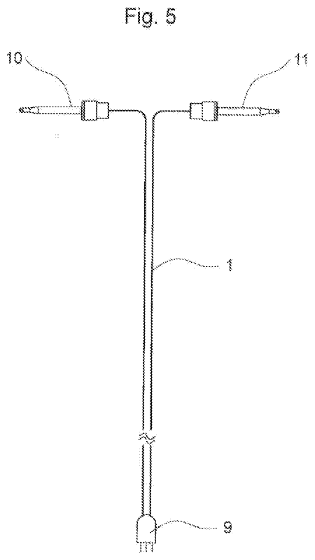

FIG. 5 shows a cable for the connection of the detachment device to a GDC implant system; and

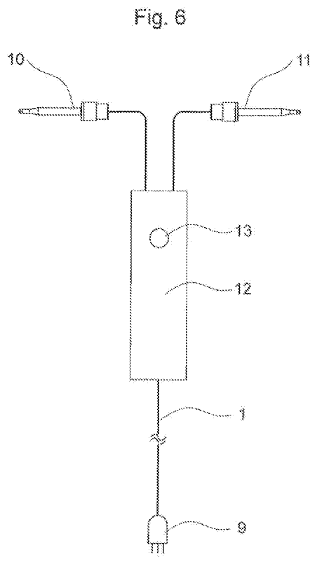

FIG. 6 illustrates the cable shown in FIG. 5 with integrated remote trigger.

FIG. 1 is a lateral view of the implant-side end of a cable 1 proposed by the present invention; the end of the cable on the implant side is formed by the insertion funnel 3, which has a lateral reinforcement 4. The insertion funnel 3 is attached to the printed circuit board 2, which is provided with spring tongues necessary to make contact with the conductive elements of the implant. Numeral 5 denotes the device-side end of cable 1, which leads to the connecting plug (not shown).

FIG. 2 is a top view of the implant-side end of the inventive cable 1 and illustrates the insertion funnel 3, the lateral reinforcements 4 as well as the device-side the end 5 of the cable 1.

FIG. 3 is the implant-side end view of cable 1 according to the invention, showing the enclosure 12, on the front side the insertion funnel 3 and the (optional) remote trigger 13, as well as the device-side end with the plug connector 9 of the cable 1.

FIG. 4 shows the connector socket 6 of the detachment device according to the invention, including five contact pins A to E, which, by way of example, are arranged concentrically inside, as well as two oppositely arranged latching elements 7, and two guiding lugs 8, which are located adjacent to each other between the two latching elements 7, with a space being left between the guiding lugs 8 and likewise spaces being arranged to the latching elements 7. The spaces between guiding lugs 8 and towards latching elements 7 are of different size, which enables a detection function with regard to the respective cable systems connected. The illustrated configuration of the connector socket allows for at least three different cable systems to be employed comprising elements complementary to the guiding lugs and latching elements. It is to be understood that guiding lugs and latching elements are arranged concentrically around the center of the socket and the connected cables make contact with the respective contact pins for the associated detachment system resulting in the required detachment current and the necessary detachment time to be triggered. It is also possible to bring about the plug coding by wiring the contacts as required. If, for example, two contacts are used for the actual current conduction, one contact for triggering the remote control, then two further contacts remain in this example which can be used for an electrical coding of the connection cable. All possible electrical signals are conceivable here, in particular level and resistance combinations.

In FIG. 5 a connection cable is displayed that is an alternative to the one depicted in FIG. 1 and can be used, for example, for monopolar GDC systems. Cable 1 is attached to the detachment device's connector socket 6 via the connecting plug 9 and terminates in two contact clamps 10 and 11, which are attached to the implant and the needle positioned in the shoulder area of the patient. Cables of this type are known in principle; however, they do not have an identifying feature that would enable them to be used together with other implant-specific cables on the same device.

FIG. 6 shows the cable illustrated in FIG. 5 with relevant connections 9, 10 and 11, as well as an enclosure 12 and, for remote actuation, a remote triggering button 13.

1. Cable for the connection of an electrical detachment device to the end of a catheter-guided implant, with the cable (1) comprising at least two current conducting wires, a connecting plug (9) for joining the cable (1) to the detachment device, and a plug connector for connection to current conducting elements of the implant, and wherein the current conducting wires being connected on a device side to contacts of the connecting plug (9) and on an implant side to contacts of the plug connector,
characterized in that
the cable (1) on the implant side being provided with an insertion funnel (3) which is suitable for conductively joining the current carrying elements of the implant to the contacts of the plug connector.

2. Cable according to claim 1, characterized in that the contacts of the plug connector are contact springs.

3. Cable according to claim 1, characterized in that the contacts of the plug connector are arranged on a printed circuit board (2).

4. Cable according to claim 1, characterized in that the cable is provided with at least two current carrying wires.

5. Cable according to claim 1, characterized in that the cable is provided with three or more current conducting wires, of which at least one is connected to a remote trigger (13) and, optionally, further wires serve for electrical coding purposes.

6. Cable according to claim 1, characterized in that the insertion funnel (3) has, on the device, a diameter which is suitably adapted to dimensions of the current carrying elements of the implant.

7. Cable according to claim 1, characterized in that the connecting plug (9) is provided with guiding elements (8) for ensuring correct connection to the detachment device as well as latching elements (7) for establishing a firm joint.

8. Detachment device for disconnection of a catheter-based implant from a guidewire, wherein the detachment device being provided with a power source, an on/off switch, a release button, signal lights to indicate an operating state and malfunctions, electronic control elements governing the detachment function as well as a connector socket (6) for connection to a cable (1) that establishes a connection to the guidewire of the catheter-guided implant,
characterized in that
the connector socket (6) has at least two contacts, latching elements (7) establishing a firm connection to complementary latching elements of the cable (1), as well as guiding elements (8) to make sure cable (1) has been correctly fitted.

9. Detachment device, characterized in that said device selects the detachment parameters in accordance with the wiring of the connector socket (6) and respective coding prerequisites of the connection cables.

10. Detachment device according to claim 8, characterized in that the connector socket (6) is a round socket comprising contact pins.

11. Detachment device according to claim 10, characterized in that the connector socket (6) is provided with at least three contact pins.

12. Detachment device according to claim 10, characterized in that the connector socket (6) is provided with five contact pins.

13. Detachment device according to claim 8, characterized in that the connector socket (6) has two latching elements (7) positioned opposite one another.

14. Detachment device according to claim 8, characterized in that the connector socket (6) is provided with two guiding elements (8).

15. Detachment device according to claim 14, characterized in that the guiding elements (8) are located adjacent to one another in such a way that they allow the connection of different cables (1) for attachment to the guidewire of the catheter-guided implant.

16. Detachment device according to claim 14, characterized in that the guiding elements (8) are guiding lugs.

17. Detachment device according to claim 10, characterized in that the latching elements (7) and guiding elements (8) are arranged concentrically in the connector socket (6).

18. Combination of a detachment device according to claim 8 for the detachment of a catheter-guided implant from a guidewire, with a cable (1) for establishing a connection with the guidewire.

19. Combination according to claim 18, characterized in that the cable (1) is a cable for the connection of an electrical detachment device to the end of a catheter-guided implant, with the cable (1) comprising at least two current conducting wires, a connecting plug (9) for joining the cable (1) to the detachment device, and a plug connector for connection to current conducting elements of the implant, and wherein the current conducting wires being connected on the device side to contacts of the connecting plug (9) and on the implant side to contacts of the plug connector, characterized in that
the cable (1) on the implant side being provided with an insertion funnel (3) which is suitable for conductively joining the current carrying elements of the implant to the contacts of the plug connector.
